(19) United States
(12) Patent Application Publication
(10) Pub. No.: US 2008/0194533 A1
(43) Pub. Date: Aug. 14, 2008

(54) PROCESS FOR SELECTIVE SULFATION OF AROMATIC HYDROXYL GROUPS

(75) Inventors: Sreenivasulu MEGATI, New City, NY (US); Panolil RAVEENDRANATH, Monroe, NY (US); Girija Raveendranath, Monroe, NY (US); Vijay Raveendranath, Albany, NY (US); Sanjay Raveendranath, Monroe, NY (US)

Correspondence Address: Pepper Hamilton LLP/Wyeth, 400 Berwyn Park, 899 Cassatt Road, Berwyn, PA, 19312-1183, US

(73) Assignee: WYETH, Madison, NJ (US)

(21) Appl. No.: 12/027,502

(22) Filed: Feb. 7, 2008

Related U.S. Application Data
(60) Provisional application No. 60/889,029, filed on Feb. 9, 2007.

(51) Int. Cl. A61K 31/566; C07J 1/00; A61P 5/24; A61P 19/08; A61P 35/00; A61P 9/00; C12N 5/06

(52) U.S. Cl. 514/182; 552/626; 435/325

(57) ABSTRACT

The present invention relates to processes for selective sulfation of an aromatic hydroxyl group over an aliphatic hydroxyl group where both are present in the same molecule. This invention also relates to processes for selective sulfation of the aromatic hydroxyl group of equilin, equilenin, estradiol, estra(1,3,5-triene)-3,16,17-triol, dihydroequilenin or dihydroequilin. This invention further relates to alkali metal salts of dihydroequilenin sulfates, dihydroequilin sulfates, estradiol sulfates, and estriol sulfates, processes for making thereof, stable compositions comprising thereof, and the use thereof.

This application claims benefit of priority to U.S. provisional patent application Ser. No. 60/889,029 filed Feb. 9, 2007, which is hereby incorporated by reference in its entirety.

### FIELD OF THE INVENTION

This invention relates to processes for selective sulfation of an aromatic hydroxyl group over an aliphatic hydroxyl group where both are present in the same molecule. This invention also relates to processes for selective sulfation of an aromatic hydroxyl group over an aliphatic hydroxyl group where both are present in the same molecule without employment of protecting groups. This invention further relates to processes for selective sulfation of the aromatic hydroxyl group of equilin, equilenin, estradiol, estra(1,3,5-triene)-3,16,17-triol, dihydroequilenin or dihydroequilin. This invention further relates to alkali metal salts of dihydroequilenin sulfates, dihydroequilin sulfates, estradiol sulfates, and estriol sulfates, processes for making thereof, stable compositions comprising thereof, and the use thereof.

### BACKGROUND OF THE INVENTION

The pleiotropic effects of estrogens in mammalian tissues have been well documented, and it is now appreciated that estrogens affect many organ systems [Mendelsohn and Karas, New England Journal of Medicine 340: 1801-1811 (1999), Epperson, et al., Psychosomatic Medicine 61: 676-697 (1999), Crandall, Journal of Womens Health & Gender Based Medicine 8: 1155-1166 (1999), Monk and Brodaty, Dementia & Geriatric Cognitive Disorders 11: 1-10 (2000), Hurn and Macrae, Journal of Cerebral Blood Flow & Metabolism 20: 631-652 (2000), Calvin, Maturitas 34: 195-210 (2000), Finking, et al., Zeitschrift fur Kardiologie 89: 442-453 (2000), Brincat, Maturitas 35: 107-117 (2000), Al-Azzawi, Postgraduate Medical Journal 77: 292-304 (2001)]. Estrogens can exert effects on tissues in several ways, and the most well characterized mechanism of action is their interaction with estrogen receptors leading to alterations in gene transcription. Estrogen receptors are ligand-activated transcription factors and belong to the nuclear hormone receptor superfamily. Other members of this family include the progesterone, androgen, glucocorticoid and mineralocorticoid receptors. Upon binding ligand, these receptors dimerize and can activate gene transcription either by directly binding to specific sequences on DNA (known as response elements) or by interacting with other transcription factors (such as AP1), which in turn bind directly to specific DNA sequences [Moggs and Orphanides, EMBO Reports 2: 775-781 (2001), Hall, et al., Journal of Biological Chemistry 276: 36869-36872 (2001), McDonnell, Principles Of Molecular Regulation. p 351-361 (2000)]. A class of “coregulatory” proteins can also interact with the ligand-bound receptor and further modulate its transcriptional activity [McKenna, et al., Endocrine Reviews 20: 321-344 (1999)]. It has also been shown that estrogen receptors can suppress NFκB-mediated transcription in both a ligand-dependent and independent manner [Quaedackers, et al., Endocrinology 142: 1156-1166 (2001), Bhat, et al., Journal of Steroid Biochemistry & Molecular Biology 67: 233-240 (1998), Pelzer, et al., Biochemical & Biophysical Research Communications 286: 1153-7 (2001)].

Estrogen receptors can also be activated by phosphorylation. This phosphorylation is mediated by growth factors such as EGF and causes changes in gene transcription in the absence of ligand [Moggs and Orphanides, EMBO Reports 2: 775-781 (2001), Hall, et al., Journal of Biological Chemistry 276: 36869-36872 (2001)].

A less well-characterized means by which estrogens can affect cells is through a so-called membrane receptor. The existence of such a receptor is controversial, but it has been well documented that estrogens can elicit very rapid non-genomic responses from cells. The molecular entity responsible for transducing these effects has not been definitively isolated, but there is evidence to suggest it is at least related to the nuclear forms of the estrogen receptors [Levin, Journal of Applied Physiology 91: 1860-1867 (2001), Levin, Trends in Endocrinology & Metabolism 10: 374-377 (1999)].

Two estrogen receptors have been discovered to date. The first estrogen receptor was cloned about 15 years ago and is now referred to as ERα [Green, et al., Nature 320: 134-9 (1986)]. The second form of the estrogen receptor was found comparatively recently and is called ERβ [Kuiper, et al., Proceedings of the National Academy of Sciences of the United States of America 93: 5925-5930 (1996)]. Early work on ERβ focused on defining its affinity for a variety of ligands and indeed, some differences with ERα were seen. The tissue distribution of ERβ has been well mapped in the rodent and it is not coincident with ERα. Tissues such as the mouse and rat uterus express predominantly ERα, whereas the mouse and rat lung express predominantly ERβ [Couse, et al., Endocrinology 138: 4613-4621 (1997), Kuiper, et al., Endocrinology 138: 863-870 (1997)]. Even within the same organ, the distribution of ERα and ERβ can be compartmentalized. For example, in the mouse ovary, ERβ is highly expressed in the granulosa cells and ERα is restricted to the thecal and stromal cells [Sar and Welsch, Endocrinology 140: 963-971 (1999), Fitzpatrick, et al., Endocrinology 140: 2581-2591 (1999)]. However, there are examples where the receptors are coexpressed and there is evidence from in vitro studies that ERα and ERβ can form heterodimers [Cowley, et al., Journal of Biological Chemistry 272: 19858-19862 (1997)].

A large number of compounds have been described that either mimic or block the activity of 17β-estradiol. Compounds having roughly the same biological effects as 17β-estradiol, the most potent endogenous estrogen, are referred to as “estrogen receptor agonists”. Those which, when given in combination with 17β-estradiol, block its effects are called “estrogen receptor antagonists”. In reality there is a continuum between estrogen receptor agonist and estrogen receptor antagonist activity and indeed some compounds behave as estrogen receptor agonists in some tissues and estrogen receptor antagonists in others. These compounds with mixed activity are called selective estrogen receptor modulators (SERMS) and are therapeutically useful agents (e.g. EVISTA) [McDonnell, Journal of the Society for Gynecologic Investigation 7: S10-S15 (2000), Goldstein, et al., Human Reproduction Update 6: 212-224 (2000)]. The precise reason why the same compound can have cell-specific effects has not been elucidated, but the differences in receptor conformation and/or in the milieu of coregulatory proteins have been suggested.

It has been known for some time that estrogen receptors adopt different conformations when binding ligands. However, the consequence and subtlety of these changes has been only recently revealed. The three dimensional structures of ERα and ERβ have been solved by co-crystallization with various ligands and clearly show the repositioning of helix 12 in the presence of an estrogen receptor antagonist which sterically hinders the protein sequences required for receptor-coregulatory protein interaction [Pike, et al., Embo 18: 4608-4618 (1999), Shiau, et al., Cell 95: 927-937 (1998)]. In addition, the technique of phage display has been used to identify peptides that interact with estrogen receptors in the presence of different ligands [Paige, et al., Proceedings of the National Academy of Sciences of the United States of America 96: 3999-4004 (1999)]. For example, a peptide was identified that distinguished between ERα bound to the full estrogen receptor agonists 17β-estradiol and diethylstilbesterol. A different peptide was shown to distinguish between clomiphene bound to ERα and ERβ. These data indicate that each ligand potentially places the receptor in a unique and unpredictable conformation that is likely to have distinct biological activities.

As mentioned above, estrogens affect a panoply of biological processes. In addition, where gender differences have been described (e.g. disease frequencies, responses to challenge, etc), it is possible that the explanation involves the difference in estrogen levels between males and females.

Compounds having estrogenic activity are disclosed in U.S. Pat. No. 6,794,403, which is incorporated herein by reference in its entirety.

The use of naturally occurring estrogenic compositions of substantial purity and low toxicity such as Premarin® has become a preferred medial treatment for alleviating the symptoms of menopausal syndrome osteoporosis/osteopenia in estrogen deficient women and in other hormone related disorders. The estrogenic components of the naturally occurring estrogenic compositions have been generally identified as sulfate esters of estrone, equilin, equilenin, β-estradiol, dihydroequilenin and β-dihydroequilenin (see, U.S. Pat. No. 2,834,712, which is incorporated herein by reference in its entirety). The incorporation of antioxidants to stabilize synthetic conjugated estrogens and the failure of pH control with Tris® to prevent hydrolysis is discussed in U.S. Pat. No. 4,154,820, which is incorporated herein by reference in its entirety. The preparation of alkali metal salts of 8,9-dehydroestrone sulfate ester is discussed in U.S. Pat. No. 5,210,081, which is incorporated herein by reference in its entirety.

The sulfation of steroid hydroxyl groups using tertiary amine-sulfur trioxide complexes (Gilbert, E. E. Chemical Reviews, 62, 1962, 549) represents a facile procedure for the preparation of steroid sulfate conjugates (Dusza, J. P. et. al.; Steroids, 12, 1968, 49 and steroids, 1985, 303). Mono-sulfation of estradiol using triethylamine-sulfur trioxide gave less than satisfactory yield of the 3-monosulfate (see, Fex, H. et. al. Acta Chemica Scandinavia, 22, 1968, 254-264) with incomplete conversions. Synthesis of estriol-3-sulfate was achieved in four steps from 16-bromo-estrone in very poor yields (see, Numazawa, M. et. al., Steroids, 1981, 557). These methods for the preparation of estrogen mono-sulfates was hampered by low to poor yields often involve multiple steps. Because improved processes for making drug molecules are consistently sought, there is an ongoing need for efficient processes for making new or existing drug molecules. The present invention is directed to this and other important ends.

### SUMMARY OF THE INVENTION

In some embodiments, the present invention provides processes for selective sulfation of an aromatic hydroxyl group over an aliphatic hydroxyl group where both are present in the same molecule. In some embodiments, the present invention provides processes for selective sulfation of an aromatic hydroxyl group over an aliphatic hydroxyl group where both are present in the same molecule without employment of protecting groups. In some embodiments, the present invention provides processes comprising:

reacting a compound of formula IIa:

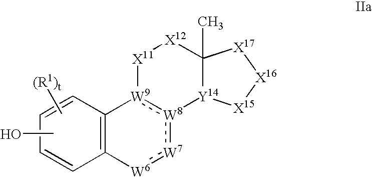

or a salt thereof, wherein:
R¹ is, at each occurrence, independently, halogen, OR^{a}, SR^{a}, —S(═O)R^{a}, —S(═O)₂R^{a}, —NO₂, —NR^{c}R^{d}, —N(R^{c})C(═O)R^{b}, —CN, —CHFCN, —CF₂CN, C₁₋₆ alkyl, C₁₋₆ haloalkyl, C₂₋₇ alkenyl, C₂₋₇ alkynyl, C₃₋₈ cycloalkyl, C₆₋₁₀ aryl, or a 5 or 6-membered heterocyclic ring having 1 to 4 heteroatoms selected from O, N and S, wherein each of the C₁₋₆ alkyl, C₂₋₇ alkenyl and C₂₋₇ alkynyl is optionally substituted by 1, 2, 3, 4 or 5 substituents independently selected from hydroxyl, —CN, —NO₂, halogen, C₁₋₆ haloalkyl, C₁₋₆ alkoxy, C₁₋₆ haloalkoxy, —C(═O)R^{b′}, —C(═O)OR^{a′}, C(═O)NR^{c′}R^{d′}, NR^{c′}R^{d′} and —N(R^{c′})C(═O)R^{b′};
[symbol] is a single bond or a double bond;
W⁶ and W⁷ are each, independently, CR⁶ or CR⁶R⁷;
R⁶ and R⁷ are each, independently, H, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
W⁸ and W⁹ are each, independently, C or CR⁸;
R³ is, at each occurrence, independently, H, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
X¹¹ and X¹² are each, independently, CR¹¹R¹²;
R¹¹ and R¹² are each, independently, H, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
Y¹⁴ is CR¹⁴;
R¹⁴ is H, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
X¹⁵, X¹⁶ and X¹⁷ are each, independently, CR¹⁵R¹⁶;
R¹⁵ and R¹⁶ are each, independently, hydrogen, hydroxyl, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
R^{a} and R^{b} are each, independently, hydrogen, C₁₋₆ alkyl, C₃₋₈ cycloalkyl or C₆₋₁₀ aryl;
R^{a′} and R^{b′} are each, independently, hydrogen, C₁₋₆ alkyl, C₃₋₈ cycloalkyl or C₆₋₁₀ aryl;
R^{c} and R^{d} are each, independently, hydrogen, C₁₋₆ alkyl, C₃₋₈ cycloalkyl or C₆₋₁₀ aryl;
or R^{c} and R^{d} together with the N atom to which they are attached form a 4-, 5-, 6- or 7-membered heterocycloalkyl group;
R^{c′} and R^{d′} are each, independently, hydrogen, C₁₋₆ alkyl, C₃₋₈ cycloalkyl or C₆₋₁₀aryl;
or R^{c′} and R^{d′} together with the N atom to which they are attached form a 4-, 5-, 6- or 7-membered heterocycloalkyl group; and
t is 0, 1, 2, or 3,
provided that at least one of X¹⁵, X¹⁶ and X¹⁷ is C(OH)R¹⁶,
   with a sulfating reagent in the presence of a base having the structure of ML wherein M is an alkali metal ion; and L is hydride (H⁻), hydroxide (OH⁻), or C₁₋₁₀ alkoxide (C₁₋₁₀ alkyl-O⁻),
   for a time and under conditions sufficient to form a compound of Formula Ia:

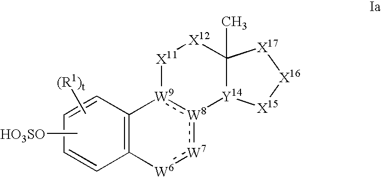

or an alkali metal salt thereof, wherein the product of the reaction of the process is substantially free of a compound of Formula XX:

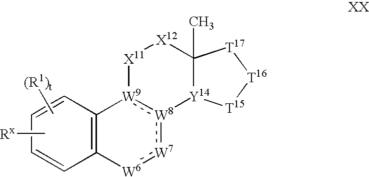

or a salt thereof, wherein R^{x} is OH or OSO₃H; and T¹⁵, T¹⁶ and T¹⁷ are each, independently, CR¹⁵R¹⁶ or C(OSO₃H)R¹⁶, and at least one of T¹⁵, T¹⁶ and T¹⁷ is C(OSO₃H)R¹⁶.

In some embodiments, Formula IIa is Formula IIaa:

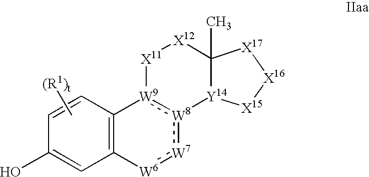

### and Formula Ia is Formula Iaa:

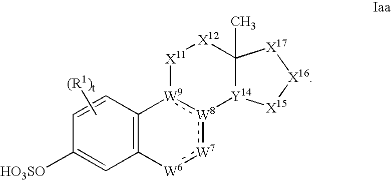

In some embodiments, Formula IIa is Formula IIab:

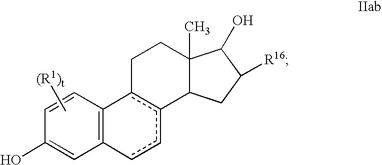

### and Formula Ia is Formula Iab:

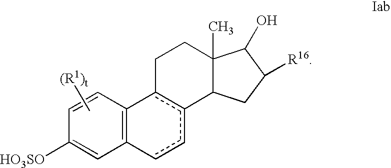

In some embodiments, the present invention provides processes for selective sulfation of the aromatic hydroxyl group of equilin, equilenin, estradiol, estra(1,3,5-triene)-3,16,17-triol, dihydroequilenin or dihydroequilin.

In some embodiments, the processes further include isolating the compound of Formula Ia or the salt thereof. In some embodiments, the processes further include adding tris(hydroxymethyl)aminomethane to the compound of Formula Ia, or the salt thereof. In some embodiments, the processes (optionally including isolating the compound of Formula Ia or the salt thereof, and optionally including adding tris(hydroxymethyl)aminomethane to the compound of Formula Ia or the salt thereof) are carried out in one reaction vessel (one-pot process).

In some embodiments, the present invention provides the compound of Formula Ia or the salt thereof, and/or a composition comprising the same. In some embodiments, the present invention provides an alkali metal salt of estra(1,3,5-triene)-3,16β,17α-triol-3-sulfate, or a composition comprising thereof. In some embodiments, the present invention provides a composition comprising an alkali metal salt of estra(1,3,5-triene)-3,16β,17α-triol-3-sulfate, and tris(hydroxymethyl)aminomethane, wherein the composition is free from other estrogenic steroids.

In some embodiments, the present invention provides a composition comprising tris(hydroxymethyl)aminomethane and a salt selected from an alkali metal salt of 17β-dihydroequilenin-3-sulfate, an alkali metal salt of 17β-dihydroequilin-3-sulfate, and an alkali metal salt of 17β-estradiol-3-sulfate (also known as 17β-dihydroestrone-3-sulfate), wherein the compositions is free from other estrogenic steroids.

In some embodiments, the present invention provides the use of the compounds and compositions described herein.

### DESCRIPTION OF THE INVENTION

In some embodiments, the present invention provides processes for selective sulfation of an aromatic hydroxyl group over an aliphatic hydroxyl group where both are present in the same molecule. In some embodiments, the present invention provides processes for selective sulfation of an aromatic hydroxyl group over an aliphatic hydroxyl group where both are present in the same molecule without employment of protecting groups.

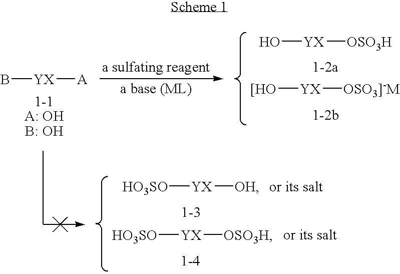

As shown in Scheme 1, compound 1-1 is a compound contains an aromatic moiety (moiety X), an aliphatic moiety (moiety Y), an aromatic hydroxyl group (A: the OH attached to the moiety X) and an aliphatic hydroxyl group (B: the OH attached to the moiety Y). An aromatic moiety in a molecule refers to an cyclic part of the molecule and the cyclic part has aromatic characters (e.g., 4n+2 delocalized electrons, and planar configuration). The aromatic moiety of such cyclic part can be optionally substituted by one or more substituents known to those skilled in the art of organic chemistry, for example, halogen, hydroxyl, alkoxy, haloalkoxy, alkyl, haloakyl, arylalkyl, amino and the like. In some embodiments, two subsituents on the aromatic moiety can be taken together to form an additional ring structure (could be mono- or poly-cyclic) which has at least two atoms common to the adjoining aromatic moiety (for example, the additional ring structure and the aromatic ring are “fused rings”). The aromatic moieties include both aryl (such as phenyl, naphthyl or the like) and heteroaryl (such as pyridyl, pyrazinyl or the like). In some embodiments, aromatic moieties include phenyl or naphthyl. In some embodiments, aromatic moieties include phenyl or naphthyl, and the phenyl or the nathphyl is fused to an additional ring system (which can be mono- or poly-cyclic). In some such embodiments, the additional ring system can further be substituted by one or more suitable substituents known to those skilled in the art of organic chemistry, for example, halogen, hydroxyl, alkoxy, haloalkoxy, alkyl, haloakyl, arylalkyl, amino and the like.

An aromatic hydroxyl group is a hydroxyl (OH) group attached to an aromatic carbon atom of an aromatic moiety. An aromatic carbon atom is a ring-forming carbon atom of the aromatic ring (such as one of the six carbon atoms in a benzene or naphthalene ring). Typically, the pKa value of an aromatic hydroxyl group is between about 3 and about 11, between about 4 and about 10, or between about 6 and about 10 (for example, pKa of phenol hydroxyl is 9.92).

An aliphatic moiety in a molecule refers to a part of the molecule which is non-aromatic, including a chain structure (including both saturated and unsaturated, straight and branched) or a cyclic structure (including mono- and poly-cyclic ring structure). The non-aromatic structure can be optionally substituted by one or more suitable substituents known to those skilled in the art of organic chemistry, for example, halogen, hydroxyl, alkoxy, haloalkoxy, alkyl, haloakyl, arylalkyl, amino and the like. Where the non-aromatic structure is a ring structure, the ring structure can further be fused to an optionally substituted aryl or heteroaryl. Non-limiting examples of aliphatic moieties include alkyl, alkenyl, cyclcoalkyl, and the like. In some embodiments, the aliphatic moieties include cyclcoalkyl groups. In some embodiments, the aliphatic moieties include optionally substituted cyclcoalkyl groups.

An aliphatic hydroxyl group is an “OH” group attached to an aliphatic carbon atom of an aliphatic moiety. In some embodiments, the aliphatic hydroxyl group is attached to a carbon atom of an alkyl group or a cycloalkyl group. In some embodiments, the aliphatic moiety to which the aliphatic hydroxyl group is attached is optionally substituted by one or more substituents. Typically, the pKa value of an aliphatic hydroxyl group is greater than about 14 (for example, pKa of ethanol hydroxyl is 15.9).

The aromatic moiety and the aliphatic moiety in the same molecule can be linked by one or more bonds. For example, where the aliphatic moiety is a ring structure, the aliphatic moiety can be fused to the aromatic moiety, or linked to the aromatic moiety through a single bond. For another example, where the aliphatic moiety is a chain structure, the aliphatic moiety can be linked to the aromatic moiety through a single bond.

Typically, the reaction of the compound 1-1 and the sulfating reagent is performed in a solvent system. The solvent system contains one or more organic solvents. A wide variety of suitable organic solvents can be employed for the solvent systems, including polar organic solvents, preferably polar aprotic organic solvents—i.e., organic solvents that are not readily deprotonated in the presence of a strongly basic reactant. Suitable aprotic solvents can include, by way of example and without limitation, ethers, halogenated hydrocarbons (e.g., a chlorinated hydrocarbon such as methylene chloride, and chloroform), N,N-dimethylformamide (DMF), N,N-dimethylacetamide (DMAC), 1,3-dimethyl-3,4,5,6-tetrahydro-2(1H)-pyrimidinone (DMPU), 1,3-dimethyl-2-imidazolidinone (DMI), N-methyl-2-pyrrolidinone (NMP, or N-methyl-2-pyrrolidone), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, or hexamethylphosphoramide. Also included within the term aprotic solvent are esters, hydrocarbons, alkylnitriles (such as acetonitrile), and many ether solvents including: dimethoxymethane, tetrahydrofuran, 2-methyl-tetrahydrofuran, 1,3-dioxane, 1,4-dioxane, furan, diethyl ether, tetrahydropyran, diisopropyl ether, dibutyl ether, ethylene glycol dimethyl ether, ethylene glycol diethyl ether, diethylene glycol dimethyl ether, diethylene glycol diethyl ether, triethylene glycol dimethyl ether, anisole, and t-butyl methyl ether. In some embodiments, the reaction is performed in a solvent system that includes or consists of an ether, for example tetrahydrofuran. In some embodiments, the solvent system can contain an alcohol (such as methanol) especially when an alkali metal alkoxide (such as sodium methoxide) is used as the base.

Typically, the compound 1-1 is dissolved in the solvent system to form a solution, and to the solution is added the base. The addition of the base is carried out at a suitable temperature (for example room temperature). In some embodiments, the solution can be optionally cooled, for example to a temperature less than about 10° C., preferably between about −10° C. and 10° C., for example about 0° C., prior to the addition of the base. Alternatively, the base can be added to the solvent system before or at the same time with the compound 1-1.

Preferably, the base is selected from strong bases [pKb of which is greater than about 10] such as metal hydrides, metal hydroxides, metal alkoxides and metal carbonates. In some embodiments, the strong base is selected from metal hydrides, metal hydroxides, and metal alkoxides. In such cases, when a molar equivalent of the strong base is mixed with the compound 1-1 or a salt thereof, greater than about 99% of the aromatic hydroxyl group is deprotonated. Generally, the sulfating reagent is employed in an amount that is about one molar equivalent to the compound 1-1 or a salt thereof. For example, the ratio of the sulfating reagent to the compound 1-1 or the salt thereof can be a value of between about 0.95 and about 1.05, for example about 0.95 to about 1.00, about 0.95 to about 0.99, about 0.95 to about 0.98, about 1.01 to about 1.05, or about 1.00 to about 1.02.

A wide variety of bases can be employed. In some embodiments, the base is an alkali metal hydride (MH⁻, wherein M is an alkali metal ion), an alkali metal hydroxide (M OH⁻, wherein M is an alkali metal ion), or an alkali metal alkoxide [M (O-alkoxide)⁻, wherein M is an alkali metal ion]. Examples of alkali metal hydrides include sodium hydride and potassium hydride. Examples of alkali metal hydroxides includes lithium hydroxide, sodium hydroxide and potassium hydroxide. Examples of alkali metal alkoxides include sodium methoxide, potassium methoxide, sodium ethoxide, potassium ethoxide, potassium tert-butoxide, sodium tert-butoxide and potassium tert-pentoxide. In some embodiments, the base is an alkali metal carbonate such as sodium carbonate and potassium carbonate. The bases can be in the form of a solution or suspension before added to or mixed with the compound 1-1, for example, sodium methoxide can be in the form of a methanolic solution, and the sodium hydride can be suspended in THF.

When a base ML [wherein M is an alkali metal ion such as Li⁺, Na⁺ or K⁺, and L is hydride (H⁻), hydroxide (OH⁻), or C₁₋₁₀ alkoxide (C₁₋₁₀ alkyl-O⁻)] is mixed with the compound 1-1, we have found that the aromatic hydroxyl group is preferentially deprotonated. Whilst the scope of the present invention should not be taken to be limited to any particular theory, it is believed that the aromatic hyroxyl group is more acidic than the aliphatic hydroxyl (i.e., they have different pKa values). Where the molar ratio of the base to the compound 1-1 is about 1:1, only the aromatic hydroxyl group is deprotonated while the aliphatic hydroxyl remains substantially intact. It is believed that the deprotonated aromatic hydroxyl group reacts more readily with a sulfating reagent to form a mono-sulfated product 1-2a and/or 1-2b (selective sulfation of the aromatic hydroxyl group). We have found that surprisingly in such a selective sulfation process, sulfation of the aliphatic hydroxyl is insubstantial (less than 10%, 5%, 4%, 3%, 2%, or 1% of the aliphatic hydroxyl group will be sulfated), thus the formation of compounds having formula HO₃SO—YX—OH (1-3) or HO₃SO—YX—OSO₃H (1-4) or their salts is insubstantial (the yield of such compounds is less than 10%, 5%, 4%, 3%, 2%, or 1% by mole). As used herein, “sulfation” or “sulfating” refers to converting an —OH group to an —OSO₃H or an —OSO₃⁻.

Thus as used herein the term “substantially free”, for example “substantially free of a compound of formula XX” means that the product of the reaction contains less than 10%, 5%, 4%, 3%, 2%, or 1% of a compound in which the aliphatic hydroxyl is sulfated (for example the compound of formula XX). Similarly, a composition according to the invention that is “substantially free” of other estrogenic steroids (particularly estrogenic steroids sulfated at one or more aliphatic hydroxyl groups) has less than 10%, 5%, 4%, 3%, 2%, or 1% of such other estrogenic steroids.

The reaction of the selective sulfation shown in Scheme 1 is advantageous, in part, because it does not involve the employment of protecting groups (which requires more steps and potentially lower yields of the mono-sulfated product 1-2a and/or 1-2b). Typically, the sulfating reagent is added to the mixture of the compound 1-1 and the base in the solvent system. Several sulfating reagents are known for sulfation of hydroxyl groups, including aromatic hydroxyl groups. See, e.g., Gilbert, E. E., “the reactions of sulfur trioxide, and of its adducts, with organic compounds”; Chemical Reviews, 62, 1962, 549-89. In some embodiments, the sulfating reagent is a complex of sulfur trioxide and a tertiary amine. In some embodiments, the sulfating reagent is a complex of sulfur trioxide and a trialkylamine (e.g. triethylamine), or a complex of sulfur trioxide and pyridine. In some embodiments, the sulfating reagent is a complex of sulfur trioxide and an amide (e.g., N,N-dimethylformamide).

The reaction of the compound of 1-1 and the sulfating reagent is performed at convenient temperature, for example less than about 100° C., less than about 80° C., from about 20° C. to about 60° C., or at room temperature. Typically, the sulfating reagent is added slowly to control temperature fluctuations. The progress of the reaction can be monitored by a variety of techniques, for example by chromatographic techniques (e.g., TLC or reverse phase HPLC). The reaction between the compound 1-1 and the sulfating reagent is complete after about 5 minutes to about 10 hours. It is advantageous to collect the sulfated product as the sulfate salt 1-2b, to prevent loss of the relatively labile sulfate group during workup and purification. Thus, in some embodiments, when the reaction between the compound of 1-1 or a salt thereof and the sulfating reagent in the presence of the base is complete, the reaction mixture is not treated with an acid.

When the reaction is complete, the compound 1-2a, or the salt thereof 1-2b, can be isolated form the reaction mixture by standard work-up procedures, for example by evaporating the residue or by precipitation (followed by filtration). In some embodiments, an anti-solvent (in which the compound 1-2a or the salt thereof 1-2b has poor solubility) such as diethylether is added to the reaction mixture to precipitate out the salt 1-2b, and the salt is collected by filtration. In some embodiments, the reaction mixture is concentrated, preferably at reduced pressure, to remove the solvents. The residue (containing the salt 1-2b) is dissolved/suspended in water or an aqueous solution. In some embodiments, a reagent that stabilizes sulfate compounds such as tris(hydroxymethyl)aminomethane can be employed at the work-up procedure. In some embodiments, the residue is dissolved/suspended in an aqueous tris(hydroxymethyl)aminomethane solution. The aqueous phase is extract with an organic solvent (e.g., diethyl ether) to remove any remaining starting materials (the compound 1-1 or its salt). The progress of the removal can be monitored by a variety of techniques, for example by chromatographic techniques (e.g., TLC or reverse phase HPLC). In some embodiments, several extractions are needed to remove the starting materials.

The aqueous phase is then separated and concentrated to afford a solid of the salt 1-2b (or a mixture of the salt 1-2b and tris(hydroxymethyl)aminomethane when the aqueous tris(hydroxymethyl)aminomethane is used). In some embodiment, lyophilization techniques are employed to afford fine powders of the salt 1-2b or the mixture of the salt 1-2b and tris(hydroxymethyl)aminomethane.

The salt 1-2b thus obtained can further be purified by any standard technique, for example by recrystallization.

In some embodiments, it is advantageous that the reaction, isolation and/or purification process are carried out in one reaction vessel (one-pot process). In some embodiments, the yield of the selective sulfation product (1-2a and/or 1-2b) is greater than about 80%, 90%, 95% or 99%. In some embodiments, the selective sulfation product (1-2a and/or 1-2b) can be isolated in high purities (i.e., substantially free of the compound 1-1 or its salt), for example, the purity of the isolated sulfation product (1-2a and/or 1-2b) is greater than about 80%, 90%, 95% or 99% by weight. In some embodiments, the isolated sulfation product (1-2a and/or 1-2b) contains less than about 10%, about 5%, about 2%, or about 1% by weight of the compound 1-1 or its salt.

The selective sulfation process described hereinabove can be utilized to selectively sulfate an aromatic hydroxyl group in a steroid which further contains one or more aliphatic hydroxyl groups. In some embodiments, the present invention provides processes comprising:

reacting a compound of formula IIa:

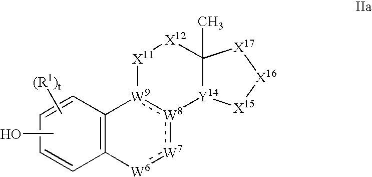

or a salt thereof, wherein:
R¹ is, at each occurrence, independently, halogen, OR^{a}, SR^{a}, —S(═O)R^{a}, —S(═O)₂R^{a}, —NO₂, —NR^{c}R^{d}, —N(R^{c})C(═O)R^{b}, —CN, —CHFCN, —CF₂CN, C₁₋₆ alkyl, C₁₋₆ haloalkyl, C₂₋₇ alkenyl, C₂₋₇ alkynyl, C₃₋₈ cycloalkyl, C₆₋₁₀ aryl, or a 5 or 6-membered heterocyclic ring having 1 to 4 heteroatoms selected from O, N and S, wherein each of the C₁₋₆ alkyl, C₂₋₇ alkenyl and C₂₋₇ alkynyl is optionally substituted by 1, 2, 3, 4 or 5 substituents independently selected from hydroxyl, —CN, —NO₂, halogen, C₁₋₆ haloalkyl, C₁₋₆ alkoxy, C₁₋₆ haloalkoxy, —C(═O)R^{b′}, —C(═O)OR^{a′}, C(═O)NR^{c′}R^{d′}, NR^{c′}R^{d′} and —N(R^{c′})C(═O)R^{b′};
[symbol] is a single bond or a double bond;
W⁶ and W⁷ are each, independently, CR⁶ or CR⁶R⁷;
R⁶ and R⁷ are each, independently, H, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
W⁸ and W⁹ are each, independently, C or CR⁸;
R⁸ is, at each occurrence, independently, H, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
X¹¹ and X¹² are each, independently, CR¹¹R¹²;
R¹¹ and R¹² are each, independently, H, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
Y⁴ is CR¹⁴;
R¹⁴ is H, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
X¹⁵, X¹⁶ and X¹⁷ are each, independently, CR¹⁵R¹⁶;
R¹⁵ and R¹⁶ are each, independently, hydrogen, hydroxyl, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
R^{a} and R^{b} are each, independently, hydrogen, C₁₋₆ alkyl, C₃₋₈ cycloalkyl or C₆₋₁₀ aryl;
R^{a′} and R^{b′} are each, independently, hydrogen, C₁₋₆ alkyl, C₃₋₈ cycloalkyl or C₆₋₁₀ aryl;
R^{c} and R^{d} are each, independently, hydrogen, C₁₋₆ alkyl, C₃₋₈ cycloalkyl or C₆₋₁₀ aryl;
or R^{c} and R^{d} together with the N atom to which they are attached form a 4-, 5-, 6- or 7-membered heterocycloalkyl group;
R^{c′} and R^{d′} are each, independently, hydrogen, C₁₋₆ alkyl, C₃₋₈ cycloalkyl or C₆₋₁₀ aryl;
or R^{c′} and R^{d′} together with the N atom to which they are attached form a 4-, 5-, 6- or 7-membered heterocycloalkyl group; and
t is 0, 1, 2, or 3,
provided that at least one of X¹⁵, X¹⁶ and X¹⁷ is C(OH)R¹⁶,
   with a sulfating reagent in the presence of a base having the structure of ML wherein M is an alkali metal ion; and L is hydride (H⁻), hydroxide (OH⁻), or C₁₋₁₀ alkoxide (C₁₋₁₀ alkyl-O⁻),
   for a time and under conditions sufficient to form a compound of Formula Ia:

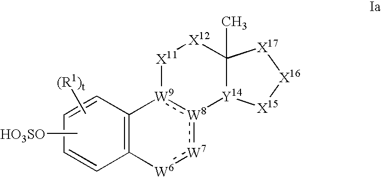

or an alkali metal salt thereof, wherein the product of the reaction of the process is substantially free of a compound of Formula XX:

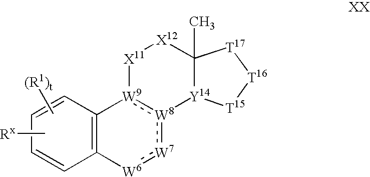

or a salt thereof, wherein R^{x} is OH or OSO₃H; and T¹⁵, T¹⁶ and T¹⁷ are each, independently, CR¹⁵R¹⁶ or C(OSO₃H)R¹⁶, and at least one of T¹⁵, T¹⁶ and T¹⁷ is C(OSO₃H)R¹⁶.

In some embodiments, Formula IIa is Formula IIaa:

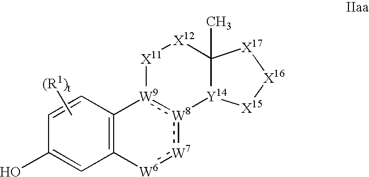

### and Formula Ia is Formula Iaa:

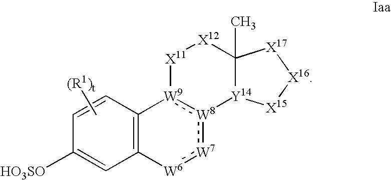

In some embodiments, Formula IIa is Formula IIab:

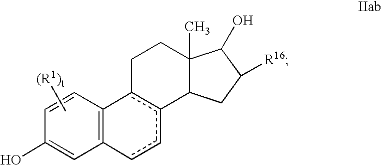

### and Formula Ia is Formula Iab:

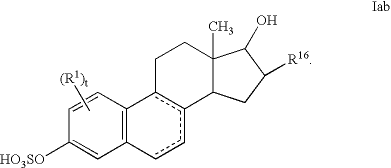

In some embodiments, the present invention provides processes for selective sulfation of the aromatic hydroxyl group of equilin, equilenin, estradiol, estra(1,3,5-triene)-3,16,17-triol, dihydroequilenin or dihydroequilenin (examples of compound of Formula IIa, IIaa or IIab).

Typically, the compound of Formula IIa or salt thereof is dissolved in a solvent system to form a solution, and to the solution is added the base. In some embodiments, the solvent system can contain one or more organic solvents, preferably polar organic solvent, more preferably polar aprotic organic solvent [such as an ether (e.g., THF, or 2-methyltetrahydrofuran), an ester (e.g., ethyl acetate), and an alkyl nitrile (e.g. acetonitrile), an amide (e.g., N,N-dimethylformamide, N,N-dimethylacetamide, N-methyl-2-pyrrolidone), or a halogenated hydrocarbon (e.g., methylene chloride, and chloroform)]. In some embodiments, the solvent system can contain an alcohol (such as methanol) especially when an alkali metal alkoxide (such as sodium methoxide) is used as the base.

The addition of the base is carried out at a suitable temperature (for example room temperature). In some embodiments, the solution can be optionally cooled, for example to a temperature less than about 10° C., preferably between about −10° C. and 10° C., for example about 0° C., prior to the addition of the base. Alternatively, the base can be added to the solvent system before or at the same time with the compound of Formula IIa.

Preferably, the base is selected from strong bases [the pKb of which is greater than about 10] such as metal hydrides, metal hydroxides, metal alkoxides and metal carbonates. In some embodiments, the strong base is selected from metal hydrides, metal hydroxides, and metal alkoxides. In such cases, when a molar equivalent of the strong base is mixed with the compound of Formula IIa or a salt thereof, greater than about 99% of the aromatic hydroxyl group is deprotonated. Generally, the sulfating reagent is employed in an amount that is about one molar equivalent to the compound of Formula IIa or salt thereof. For example, the ratio of the sulfating reagent to the compound of Formula IIa or the salt thereof can be a value of between about 0.95 and about 1.05, for example about 0.95 to about 1.00, about 0.95 to about 0.99, about 0.95 to about 0.98, about 1.01 to about 1.05, or about 1.00 to about 1.02.

A wide variety of bases can be employed. In some embodiments, the base is an alkali metal hydride (M H⁻, wherein M is an alkali metal ion), an alkali metal hydroxide (M OH⁻, wherein M is an alkali metal ion), or an alkali metal alkoxide [M (O-alkoxide)⁻, wherein M is an alkali metal ion]. Examples of alkali metal hydrides include sodium hydride and potassium hydride. Examples of alkali metal hydroxides includes lithium hydroxide, sodium hydroxide and potassium hydroxide. Examples of alkali metal alkoxides include sodium methoxide, potassium methoxide, sodium ethoxide, potassium ethoxide, potassium tert-butoxide, sodium tert-butoxide and potassium tert-pentoxide. In some embodiments, the base is an alkali metal carbonate such as sodium carbonate and potassium carbonate. The bases can be in the form of a solution or suspension before added to or mixed with the compound of Formula IIa, for example, sodium methoxide can be in the form of a methanolic solution, and the sodium hydride can be suspended in THF. In some embodiments, M is Li⁺, Na⁺ or K⁺. In some embodiments, M is Na⁺ or K⁺. In some embodiments, M is Na⁺. In some embodiments, M is K⁺.

Typically, the sulfating reagent is added to the mixture of the compound of Formula IIa or salt thereof and the base in the solvent system. In some embodiments, the sulfating reagent is a complex of sulfur trioxide and a tertiary amine. In some embodiments, the sulfating reagent is a complex of sulfur trioxide and a trialkylamine (e.g. triethylamine), or a complex of sulfur trioxide and pyridine. In some embodiments, the sulfating reagent is a complex of sulfur trioxide and an amide (e.g., N,N-dimethylformamide).

The reaction of the compound of Formula IIa or salt thereof and the sulfating reagent is performed at a convenient temperature, for example less than about 100° C., less than about 80° C., less than about 60° C., less than about 40° C., less than about 20° C., less than about 0° C., from about −20° C. to about 0° C., from about 0° C. to about 20° C., from about 20° C. to about 60° C., from about 20° C. to about 40° C., or at room temperature. Typically, the sulfating agent is added slowly to control temperature fluctuations. The progress of the reaction can be monitored by a variety of techniques, for example by chromatographic techniques (e.g., TLC or reverse phase HPLC). The reaction between the compound of Formula IIa or salt thereof and the sulfating reagent is complete after about 5 minutes to about 10 hours. It is advantageous to collect the sulfated product as a sulfate salt of the compound of Formula IIa, to prevent loss of the relatively labile sulfate group during workup and purification. Thus, in some embodiments, when the reaction between the compound of Formula IIa or salt thereof and the sulfating reagent in the presence of the base is complete, the reaction mixture is not treated with an acid.

In such a selective sulfation process, sulfation of the aliphatic hydroxyl is insubstantial (less than 10%, 5%, 4%, 3%, 2%, or 1% of the aliphatic hydroxyl group will be sulfated), thus the formation of compounds having Formula XX or salt thereof is insubstantial (the yield of such compounds/salts is less than 10%, 5%, 4%, 3%, 2%, or 1% by mole). The reaction in the selective sulfation process of the present invention is also advantageous, in part, because it does not involve the employment of protecting groups [which requires more steps (i.e., protecting and deprotecting steps) and potentially lower yields of the mono-sulfated product: the salt of the compound of Formula Ia].

When the reaction is complete, the compound of Formula Ia or the salt thereof (the salt of the compound of Formula Ia where no acid is added the reaction mixture), can be isolated form the reaction mixture by standard work-up procedures, for example by evaporating the reaction mixture to obtain a residue or by precipitation followed by filtration. In some embodiments, an anti-solvent such as diethylether is added to the reaction mixture to precipitate out the salt of the compound of Formula Ia and the salt is collected by filtration.

In some embodiments, the reaction mixture is concentrated, preferably at reduced pressure, to remove the solvents. The residue (containing the salt of the compound of Formula Ia) is dissolved/suspended in water. In some embodiments, a reagent that stabilizes sulfate compounds such as tris(hydroxymethyl)aminomethane can be employed at the work-up procedure. In some embodiments, the residue is dissolved/suspended in an aqueous tris(hydroxymethyl)aminomethane solution. The aqueous phase is extracted with an organic solvent (e.g., diethyl ether) to remove any remaining starting materials (the compound of Formula IIa or its salt). The progress of the removal can be monitored by a variety of techniques, for example by chromatographic techniques (e.g., TLC or reverse phase HPLC). In some embodiments, several extractions are needed to remove the starting materials.

The aqueous phase is then separated and concentrated to afford a solid of the salt of the compound of Formula Ia (or a mixture of the salt of the compound of Formula Ia and tris(hydroxymethyl)aminomethane when the aqueous tris(hydroxymethyl)aminomethane is used). In some embodiment, lyophilization techniques are employed to afford fine powders of the salt of the compound of Formula Ia or the mixture of the salt of the compound of Formula Ia and tris(hydroxymethyl)aminomethane.

The salt of the compound of Formula Ia thus obtained can be further purified by any standard technique, for example by recrystallization.

In some embodiments, the present invention provides an alkali metal salt (such as sodium salt) of estra(1,3,5-triene)-3,16β,17α-triol-3-sulfate, or a composition thereof. In some embodiments, the present invention provides compositions containing an alkali metal salt of estra(1,3,5-triene)-3,16β,17α-triol-3-sulfate and tris(hydroxymethyl)aminomethane, wherein the composition is free from other estrogenic steroids. In some embodiments, the present invention provides compositions containing an alkali metal salt (such as sodium salt) of 17β-dihydroequilenin-3-sulfate and tris(hydroxymethyl)aminomethane, wherein the composition is free from other estrogenic steroids. In some embodiments, the present invention provides compositions containing an alkali metal salt (such as sodium salt) of 17β-dihydroequilin-3-sulfate and tris(hydroxymethyl)aminomethane, wherein the composition is free from other estrogenic steroids. In some embodiments, the present invention provides compositions containing an alkali metal salt (such as sodium salt) of 17β-estradiol-3-sulfate (also known as 17β-dihydroestrone-3-sulfate) and tris(hydroxymethyl)aminomethane, wherein the composition is free from other estrogenic steroids.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used in this application, the term “optionally substituted,” as used herein, means that substitution is optional and therefore it is possible for the designated atom or moiety to be unsubstituted. In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example, if a methyl group (i.e., CH₃) is optionally substituted, then 3 hydrogens on the carbon atom can be replaced. Examples of suitable substituents include, but are not limited to: halogen, CN, NH₂, OH, SO, SO₂, COOH, OC₁₋₆ alkyl CH₂OH, SO₂H, C₁₋₆alkyl, OC₁₋₆ alkyl C(═O)C₁₋₆ alkyl C(═O)O—C₁₋₆ alkyl C(═O)NH₂, C(═O)NHC₁₋₆ alkyl C(═O)N(C₁₋₆ alkyl)2, SO₂C₁₋₆ alkyl, SO₂NH—C₁₋₆ alkyl SO₂N(C₁₋₆ alkyl)₂, NH(C₁₋₆alkyl), N(C₁₋₆ alkyl)₂, NHC(═O)C₁₋₆ alkyl, NC(═O)(C₁₋₆ alkyl)₂, aryl, O-aryl, C(═O)-aryl, C(═O)O-aryl, C(═O)NH-aryl, C(═O)N(aryl)₂, SO₂-aryl, SO₂NH-aryl, SO₂N(aryl)₂, NH(aryl), N(aryl)₂, NC(═O)aryl, NC(═O)(aryl)₂, heterocyclyl, O-heterocyclyl, C(═O)-heterocyclyl, C(═O)O-heterocyclyl, C(═O)NH-heterocyclyl, C(═O)N(heterocyclyl)₂, SO₂-heterocyclyl, SO₂NH-heterocyclyl, SO₂N(heterocyclyl)₂, NH(heterocyclyl), N(heterocyclyl)₂, NC(═O)-heterocyclyl, and NC(═O)(heterocyclyl)₂, or any subset thereof.

The carbon number, as used in the definitions herein, refers to carbon backbone and carbon branching, but does not include carbon atoms of substituents, such as alkoxy substitutions and the like.

As used herein, the term “alkyl” is meant to refer to a monovalent or divalent saturated hydrocarbon group which is straight-chained or branched. Example alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. An alkyl group can contain from 1 to about 20, from 2 to about 20, from 1 to about 10, from 1 to about 8, from 1 to about 6, from 1 to about 4, or from 1 to about 3 carbon atoms, or if a specified number of carbon atoms is provided then that specific number would be intended. For example “C₁₋₆ alkyl” denotes alkyl having 1, 2, 3, 4, 5 or 6 carbon atoms. As used herein, the term “lower alkyl” is intended to mean alkyl groups having up to six carbon atoms.

As used herein, “alkenyl” refers to an alkyl group having one or more carbon-carbon double bonds. Nonlimiting examples of alkenyl groups include ethenyl, propenyl, and the like.

As used herein, “alkynyl” refers to an alkyl group having one or more carbon-carbon triple bonds. Nonlimiting examples of alkynyl groups include ethynyl, propynyl, and the like.

As used herein, “aromatic” refers to having the characters such as 4n+2 delocalized electrons in a ring structure and planar configuration of the ring.

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be a polycyclic moiety in which at least one carbon is common to any two adjoining rings therein (for example, the rings are “fused rings”), for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls or cycloalkynyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, “cycloalkyl” refers to non-aromatic cyclic hydrocarbons including cyclized alkyl, alkenyl, and alkynyl groups, having the specified number of carbon atoms (wherein the ring comprises 3 to 20 ring-forming carbon atoms). Cycloalkyl groups can include mono- or polycyclic (e.g., having 2, 3 or 4 fused or bridged rings) groups. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like, or any subset thereof. Also included in the definition of cycloalkyl are moieties that have one or more aromatic rings fused (i.e., having a bond in common with) to the cycloalkyl ring, for example, benzo derivatives of cyclopentane (i.e., indanyl), cyclopentene, cyclohexane, and the like. The term “cycloalkyl” further includes saturated ring groups, having the specified number of carbon atoms. These may include fused or bridged polycyclic systems. Suitable cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, the term “heterocyclyl” or “heterocyclic” or “heterocycle” refers to ring-containing monovalent and divalent structures having one or more heteroatoms, independently selected from N, O and S, as part of the ring structure and comprising from 3 to 20 atoms in the rings, or 3- to 7-membered rings. Heterocyclic groups may be saturated or partially saturated or unsaturated, containing one or more double bonds, and heterocyclic groups may contain more than one ring as in the case of polycyclic systems. The heterocyclic rings described herein may be substituted on carbon or on a heteroatom atom if the resulting compound is stable. If specifically noted, nitrogen in the heterocyclyl may optionally be quaternized. It is understood that when the total number of S and O atoms in the heterocyclyl exceeds 1, then these heteroatoms are not adjacent to one another.

Examples of heterocyclyls include, but are not limited to, 1H-indazole, 2-pyrrolidonyl, 2H, 6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azabicyclo, azetidine, azepane, aziridine, azocinyl, benzimidazolyl, benzodioxol, benzofuranyl, benzothiofuranyl, benzothiophenyl, benzoxazolyl, benzthiazolyl, benzotriazolyl, benzotetrazolyl, benzisoxazolyl, benzisothiazolyl, benzimidazalonyl, carbazolyl, 4aH-carbazolyl, b-carbolinyl, chromanyl, chromenyl, cinnolinyl, diazepane, decahydroquinolinyl, 2H,6H-1,5,2-dithiazinyl, dioxolane, furyl, 2,3-dihydrofuran, 2,5-dihydrofuran, dihydrofuro[2,3-b]tetrahydrofuran, furanyl, furazanyl, homopiperidinyl, imidazolidine, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxirane, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, purinyl, pyranyl, pyrrolidinyl, pyrroline, pyrrolidine, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolidinyl dione, pyrrolinyl, pyrrolyl, pyridine, quinazolinyl, quinolinyl, 4H-quinolizinyl, quinoxalinyl, quinuclidinyl, carbolinyl, tetrahydrofuranyl, tetramethylpiperidinyl, tetrahydroquinoline, tetrahydroisoquinolinyl, thiophane, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiopheneyl, thiirane, triazinyl, 1,2,3-triazolyl, 1,2,4-triazolyl, 1,2,5-triazolyl, 1,3,4-triazolyl, and xanthenyl, or any subset thereof.

As used herein, “heteroaryl” refers to an aromatic heterocycle (wherein the ring comprises up to about 20 ring-forming atoms) having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like, or any subset thereof. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl group has 1 to about 4, 1 to about 3, or 1 to 2 heteroatoms. In some embodiments, the heteroaryl group has 1 heteroatom.

As used herein, “heterocycloalkyl” refers to non-aromatic heterocycles (wherein the ring comprises about 3 to about 20 ring-forming atoms) including cyclized alkyl, alkenyl, and alkynyl groups where one or more of the ring-forming carbon atoms is replaced by a heteroatom such as an O, N, or S atom. Hetercycloalkyl groups can be mono or polycyclic (e.g., fused-, bridged- and spiro-systems). Suitable “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Ring-forming carbon atoms and heteroatoms of a heterocycloalkyl group can be optionally substituted by oxo or sulfido. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. In some embodiments, the heterocycloalkyl group has from 1 to about 20 carbon atoms, and in further embodiments from about 3 to about 20 carbon atoms. In some embodiments, the heterocycloalkyl group contains 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy and propargyloxy, or any subset thereof. Similarly, “alkylthio” or “thioalkoxy” represent an alkyl group as defined above with the indicated number of carbon atoms attached through a sulphur bridge.

As used herein, “halo” or “halogen” includes fluoro, chloro, bromo, and iodo, or any subset thereof.

As used herein, “haloalkyl” refers to an alkyl group having one or more halogen substituents. Example haloalkyl groups include CF₃, C₂F₅, CH₂CF₃, CHF₂, CCl₃, CHCl₂, C₂Cl₅, and the like, or any subset thereof. The term “perhaloalkyl” is intended to denote an alkyl group in which all of the hydrogen atoms are replaced with halogen atoms. One example of perhaloalkyl is CH₃ or CF₃. The term “perfluoroalkyl” is intended to denote an alkyl group in which all of the hydrogen atoms are replaced with fluorine atoms. One example of perhaloalkyl is CF₃ (i.e., trifluoromethyl).

As used herein, “alkoxy” or “alkyloxy” refers to an —O-alkyl group. Example alkoxy groups include methoxy, ethoxy, propoxy (e.g., n-propoxy and isopropoxy), t-butoxy, and the like, or any subset thereof.

As used here, “haloalkoxy” refers to an —O-haloalkyl group. An example haloalkoxy group is OCF₃.

As used herein, the term “reacting” refers to the bringing together of designated chemical reactants such that a chemical transformation takes place generating a compound different from any initially introduced into the system. Reacting can take place in the presence or absence of solvent.

The compounds of the present invention can contain an asymmetric atom, and some of the compounds can contain one or more asymmetric atoms or centers, which can thus give rise to optical isomers (enantiomers) and diastereomers. The present invention includes such optical isomers (enantiomers) and diastereomers (geometric isomers), as well as, the racemic and resolved, enantiomerically pure R and S stereoisomers, as well as, other mixtures of the R and S stereoisomers and pharmaceutically acceptable salts thereof. Optical isomers can be obtained in pure form by standard procedures known to those skilled in the art, and include, but are not limited to, diastereomeric salt formation, kinetic resolution, and asymmetric synthesis. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds of the invention can also include tautomeric forms, such as keto-enol tautomers. Tautomeric forms can be in equilibrium or sterically locked into one form by appropriate substitution.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography (TLC).

The reactions of the processes described herein can be carried out in air or under an inert atmosphere. Typically, reactions containing reagents or products that are substantially reactive with air can be carried out using air-sensitive synthetic techniques that are well known to the skilled artisan.

Upon carrying out preparation of compounds according to the processes described herein, the usual isolation and purification operations such as concentration, precipitation, filtration, extraction, solid-phase extraction, recrystallization, chromatography, and the like may be used to isolate the desired products.

### Methods of Use and Pharmaceutical Formulations

As described in U.S. Pat. Nos. 2,834,712, 4,154,820, and, 5,210,081, alkali metal sulfate salts of a synthetic conjugated estrogen such as selected from the group of estrone, equilin, 17α-dihydroequilin, 17β-hydroequilin, 17β-estradiol, 17α-estradiol, equilinen, and 17β-dihydroequilenin, or compositions thereof can be used for treating or preventing the disease states or syndromes associated with an estrogen deficiency or an excess of estrogen, such as menopausal syndrome, female hypogonadism, amenorrhea, female castration, primary ovarian failure, abnormal uterine bleeding due to hormonal imbalance, and senile vaginitis. Accordingly, the compounds (including the salts) and the compositions of the present invention can find many uses related to treating or preventing disease states or syndromes associated with an estrogen deficiency or an excess of estrogen. They may also be used in methods of treatment for diseases or disorders which result from proliferation or abnormal development, actions or growth of endometrial or endometrial-like tissues.

Examples of maladies which result from estrogen effects and estrogen excess or deficiency include osteoporosis, prostatic hypertrophy, male pattern baldness, vaginal and skin atrophy, acne, dysfunctional uterine bleeding, endometrial polyps, benign breast disease, uterine leiomyomas, adenomyosis, ovarian cancer, infertility, breast cancer, endometriosis, endometrial cancer, polycystic ovary syndrome, cardiovascular disease, contraception, Alzheimer's disease, cognitive decline and other CNS disorders, as well as certain cancers including melanoma, prostrate cancer, cancers of the colon, CNS cancers, among others. Additionally, the compounds (including the salts) and the compositions of the present invention can be used for contraception in pre-menopausal women, as well as hormone replacement therapy in post-menopausal women (such as for treating vasomotor disturbances such as hot flush) or in other estrogen deficiency states where estrogen supplementation would be beneficial. The compounds (including the salts) and the compositions of the present invention can further be used in disease states where amenorrhea is advantageous, such as leukemia, endometrial ablations, chronic renal or hepatic disease or coagulation diseases or disorders.

The compounds (including the salts) and the compositions of the present invention can also be used in methods of treatment for and prevention of bone loss, which can result from an imbalance in a individual's formation of new bone tissues and the resorption of older tissues, leading to a net loss of bone. Such bone depletion results in a range of individuals, particularly in post-menopausal women, women who have undergone bilateral oophorectomy, those receiving or who have received extended corticosteroid therapies, those experiencing gonadal dysgenesis, and those suffering from Cushing's syndrome. Special needs for bone, including teeth and oral bone, replacement can also be addressed using the solid dispersion in individuals with bone fractures, defective bone structures, and those receiving bone-related surgeries and/or the implantation of prosthesis. In addition to the problems described above, the compounds (including the salts) and the compositions of the present invention can be used in treatments for osteoarthritis, hypocalcemia, hypercalcemia, Paget's disease, osteomalacia, osteohalisteresis, multiple myeloma and other forms of cancer having deleterious effects on bone tissues.

Methods of treating the diseases and syndromes listed herein are understood to involve administering to an individual in need of such treatment a therapeutically effective amount of the salt form or solid dispersion of the invention, or composition containing the same. As used herein, the term “treating” in reference to a disease is meant to refer to preventing, inhibiting and/or ameliorating the disease.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomatology of the disease;

(2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and

(3) ameliorating the disease; for example, ameliorating a disease, condition or disorder in an individual that is experiencing or displaying the pathology or symptomatology of the disease, condition or disorder (i.e., reversing the pathology and/or symptomatology).

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. Effective administration of the compounds (including the salts) and the compositions of the present invention may be given at an oral dose of from about 0.1 mg/day to about 1,000 mg/day. Preferably, administration will be from about 10 mg/day to about 600 mg/day, more preferably from about 50 mg/day to about 600 mg/day, in a single dose or in two or more divided doses. The projected daily dosages are expected to vary with route of administration.

Such doses may be administered in any manner useful in directing the active compounds herein to the recipient's bloodstream, including orally, via implants, parentally (including intravenous, intraperitoneal, intraarticularly and subcutaneous injections), rectally, intranasally, topically, ocularly (via eye drops), vaginally, and transdermally.

Oral formulations containing the active compounds (including the salts) and the compositions of the present invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

In some cases it may be desirable to administer the compounds (including the salts) and the compositions of the present invention directly to the airways in the form of an aerosol.

The compounds (including the salts) and the compositions of the present invention may also be administered parenterally or intraperitoneally. Solutions or suspensions of these active compounds (including the salts) and the compositions of the present invention can be prepared in water optionally mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### Preparation of Sodium 17α-estradiol-3-sulfate (Using NaH and Et₃N—SO₃)

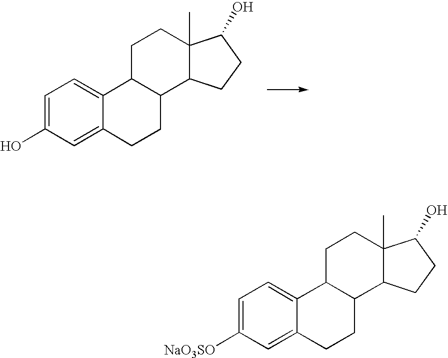

To a suspension of NaH (60%, 0.015 g, 0.36 mmol) in THF (2 ml) under N₂ atmosphere was added the 17α-estradiol (0.1 g, 0.36 mmol). After 1 h stirring at 22° C., SO₃—NEt₃ complex (0.074 g, 0.4 mmol) was added at 22° C. After the reaction was complete (monitored by HPLC), diethyl ether (3 ml) was added dropwise to the stirred reaction mixture. The precipitated white solid was filtered washed with diethyl ether (3 ml×2) to give the sodium-17α-estradiol-3-sulfate (0.12 g, yield 90%).

¹H-NMR (300 MHz, DMSO-d₆): δ 7.16 (d, 1H, J=8.4 Hz), 6.87 (m, 2H), 4.36 (d, 1H, J=4.2 Hz), 3.59 (m, 1H), 2.70-2.80 (m, 2H), 2.26-2.37 (m, 1H), 1.95-2.17 (m, 2H), 1.66-1.90 (m, 3H), 1.13-1.63 (m, 7H), 0.62 (s, 3H).

ESI LC/MS m/z 351 (M⁻, less Na).

HPLC (purity area %): 97.4%.

### Example 2

### Preparation of Sodium 17α-estradiol-3-sulfate (Using NaOMe and Me₃N—SO₃)

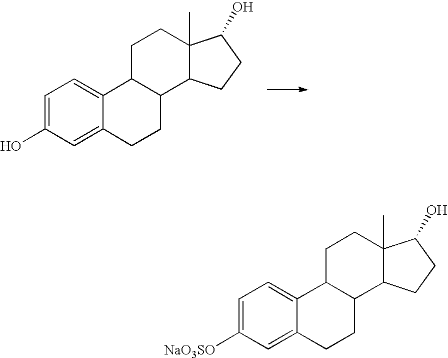

To a solution of 17α-estradiol (0.2 g, 0.73 mmol) in anhydrous THF (3 ml) was added NaOMe (30% methanolic solution, 0.14 ml, 0.73 mmol) dropwise at 22° C. Stirred for 0.5 h, then Me₃N—SO₃ (0.122 g, 0.87 mmol) was added at 22° C. After 20 h at 22° C., diethyl ether (5 ml) was added dropwise to the stirred reaction mixture. The white solid was filtered, washed with diethyl ether (5 ml×2) and dried to give sodium-17α-estradiol-3-sulfate (0.235 g, yield 86%).

¹H-NMR (300 MHz, DMSO-d₆): δ 7.16 (d, 1H, J=8.4 Hz), 6.87 (m, 2H), 4.36 (d, 1H, J=4.2 Hz), 3.59 (m, 1H), 2.70-2.80 (m, 2H), 2.26-2.37 (m, 1H), 1.95-2.17 (m, 2H), 1.66-1.90 (m, 3H), 1.13-1.63 (m, 7H), 0.62 (s, 3H).

ESI LC/MS m/z 351 (M⁻, less Na).

HPLC (purity area %) 98.2%.

### Example 3

### Preparation of Sodium 17α-dihydroequilin-3-sulfate (using NaOMe and Et₃N—SO₃)

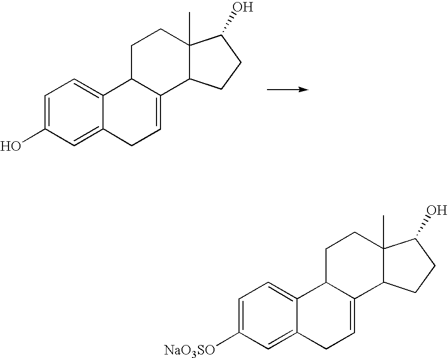

To a solution of 17α-dihydroequilin (0.5 g, 1.84 mmol) in anhydrous THF (5 ml) was added NaOMe (30% methanolic solution, 0.35 ml, 1.84 mmol) dropwise at 22° C. Stirred for 0.5 h, then Et₃N—SO₃ (0.355 g, 1.84 mmol) was added at 22° C. After 2 h at 22° C., diethyl ether (20 ml) was added dropwise to the stirred reaction mixture. The white solid was filtered, washed with diethyl ether (10 ml×2) and dried. The product was dissolved in water (deionized, 100 ml) extracted with diethyl ether (10 ml×4) and the aqueous solution was lyophilized to give sodium-17α-dihydroequilin-3-sulfate (0.58 g, yield 85%) as a white solid.

¹H-NMR (300 MHz, DMSO-d₆): δ 7.15 (d, 1H, J=8.5 Hz), 6.94 (dd, 1H, J=8.5 & 2.4 Hz), 6.88 (d, 1H, J=2.4 Hz), 5.37 (bs, 1H), 4.49 (d, 1H, J=4.23 Hz), 3.64 (t, 1H), 3.28-3.40 (m, 2H), 2.30-2.40 (m, 1H), 2.04-2.20 (m, 2H), 1.88-2.0 (m, 1H), 1.33-1.60 (m, 5H), 0.49 (s, 3H).

ESI LC/MS m/z 349 (M⁻, less Na).

HPLC (purity area %): 97.6%.

### Example 4

### Preparation of Sodium-Estra-1,3,5(10)-triene-3,16β,17α-triol-3-sulfate (using NaOMe and Et₃N—SO₃)

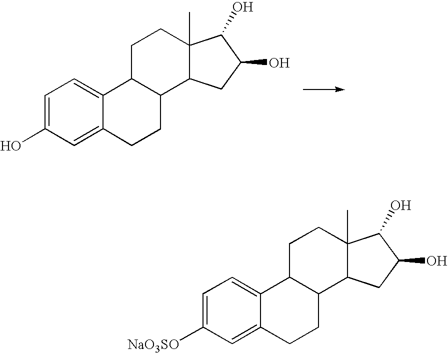

To a solution of the 3,16β,17α-estratriol (0.15 g, 0.52 mmol) anhydrous THF (5 ml) was added NaOMe (30% methanolic solution, 0.1 ml, 0.52 mmol) dropwise at 22° C. Stirred at 22° C. for 15 min, Et₃N—SO₃ (0.094 g, 0.52 mmol) was added at 22° C. After 1 h at 22° C., THF was evaporated. The residue product was dissolved in water (deionized, 20 ml) extracted with diethyl ether (20 ml×3) and the aqueous solution was lyophilized to give sodium-1,3,5(10)-estratriene-3,16β,17α-triol-3-sulfate (0.164 g, yield 82%) as a white solid.

¹H-NMR (300 MHz, DMSO-d₆): δ 7.16 (d, 1H, J=8.4 Hz), 6.87 (m, 2h), 4.78 (d, 1H, J=3.9 Hz), 4.48 (d, 1H, J=4.5 Hz), 3.83 (m, 1H), 3.32 (d, 1H), 2.78 (m, 2H), 2.28 (m, 1H), 2.08 (m, 2H), 1.72-1.85 (m, 1H), 1.63-1.71 (m, 1H), 1.23-1.55 (m, 5H), 1.08-1.27 (m, 1H), 0.82 (s, 3H).

ESI LC/MS m/z 367 (M⁻, less Na).

HPLC (purity area %): 98.8%.

### Example 5

### Preparation of Sodium-17β-dihydroequilin-3-sulfate

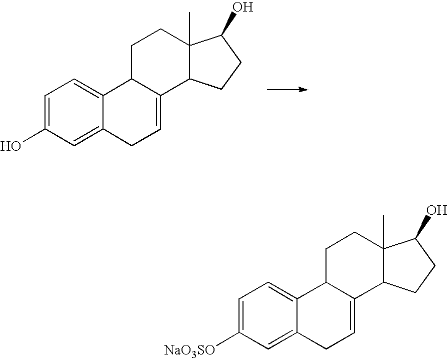

To a solution of 17β-dihydroequilin (10 g, 36.98 mmol) in THF (100 ml) at 22° C. was added 30% NaOMe/MeOH (7.0 ml, 36.98 mmol) dropwise (3 min). During the addition, clear colorless solution changed to very light pink solution. After 15 min stirring, at 22° C., sulfur trioxide-triethylamine complex (6.70 g, 36.98 mmol) was added as solid (reaction mixture self heated slightly from 22° C.-24° C.). Reaction was monitored by reverse phase HPLC. After 30 min, the solvents were evaporated from the reaction mixture. To the residual solid was added a solution of tris (hydroxymethyl) aminomethane (4.55 g, 37.46 mmol) in water (800 ml, deionized). The aqueous solution was extracted with diethyl ether (200 ml×4) till no starting material seen in the HPLC. Concentrated the aqueous layer by 10% on a rotary evaporator (to remove any residual ether), added an equivalent amount of water and the concentration was repeated once more. Finally, the aqueous solution was further diluted to a volume of 2.0 L and then lyophilized to give 16.9 g of the 3-sulfate (yield 90%).

¹H-NMR (300 MHz, DMSO-d₆): δ 7.14 (d, 1H, J=8.5 Hz), 6.95 (dd, 1H, J=8.5 & 2.5 Hz), 6.89 (d, 1H, J=2.5 Hz), 5.34 (bs, 1H), 4.62 (d, 1H, J=4.8 Hz), 3.67 (m, 1H), 3.33 (m, 2H), 3.04 (m, 1H), 2.13-2.16 (m. 1H), 1.93-1.97 (m, 2H), 1.81-1.84 (m, 1H), 1.63-1.69 (m, 1H), 1.34-1.45 (m, 4H), 0.53 (s, 3H); TRIS peaks: 4.34 (bh, 3H), 3.21 (bs, 6H), 1.24 (bs, 2H).

¹³C-NMR (300 MHz, DMSO-d₆): δ 151.47, 137.44, 133.53, 132.83, 128.27, 119.94, 119.18, 113.72, 80.53, 49.94, 45.21, 37.55, 32.70, 30.11, 29.57, 20.71, 11.80; TRIS peaks: 63.71, 57.02

ESI MS m/z 349 (M⁻, less Na).

IR (KBr): 3600-3200, 2938, 2876, 1631, 1610, 1589, 1496, 1254, 1050, 1023 cm⁻¹

HPLC (purity area %): 98.0%.

### Example 6

### Preparation of Sodium-17β-dihydroequilenin-3-sulfate

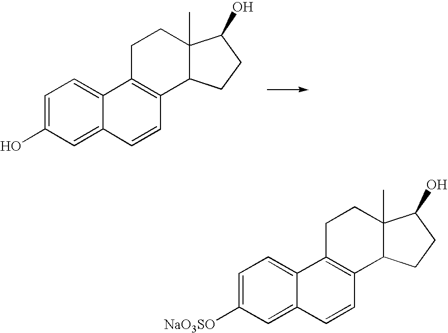

To a solution of 17β-dihydroequilenin (10 g, 37.26 mmol) in THF (100 ml) at 22° C. (RT) was added 30% NaOMe/MeOH (7.1 ml, 37.26 mmol) dropwise (3 min). During the addition, the clear solution changed to light yellow color. After 15 min stirring, at 22° C., sulfur trioxide-triethylamine complex (6.75 g, 37.26 mmol) was added (reaction mixture self heated slightly from 22° C.-24° C.). Reaction was monitored by reverse phase HPLC. After 45 min the reaction mixture was concentrated to dryness using a rotary evaporator. To the residual solid was added a solution of tris(hydroxymethyl)aminomethane (4.55 g, 37.46 mmol) in water (800 ml, deionized). The resulting aqueous solution was extracted with diethyl ether (200 ml×4) till no starting material was seen by HPLC. Solids separated out when the aqueous layer was being concentrated to remove residual ether. Water (800 ml) was added to dissolve the solid. Concentrated the aqueous layer by 10% on a rotary evaporator, added an equivalent amount of water and the concentration was repeated once more. Finally, the aqueous solution was further diluted to a volume of 2.0 L and then lyophilized to give 18.2 g of the product (yield 98%).

¹H-NMR (300 MHz, DMSO-d₆): δ 7.87 (d, 1H, J=9.2 Hz), 7.60 (m, 2H), 7.35 (dd, 1H, J=9.2 & 2.4 Hz), 7.14 (d, 1H, J=8.4 Hz), 4.69 (d, 1H, J=4.7 Hz), 3.75 (m, 1H), 3.14 (m, 2H), 2.73 (m, 1H), 2.06-2.17 (m, 3H), 1.53-1.70 (m, 3H), 0.55 (s, 3H); TRIS peaks: 4.33 (bs), 3.22 (bs), 1.27 (bs).

¹³C-NMR (300 MHz, DMSO-d₆): δ 150.52, 135.36, 132.54, 130.42, 128.61, 125.67, 125.20, 124.26, 121.72, 117.10, 79.36, 46.21, 42.77, 33.87, 30.82, 24.28, 23.48, 10.79; TRIS peaks: 63.72, 57.04.

ESI MS m/z 347 (M⁻, less Na).

HPLC (purity area %): 99.4%.

### Example 7

### Preparation of Sodium 17β-dihydroequilenin-3-sulfate

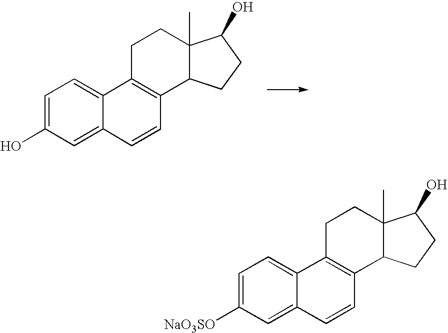

To a solution of 17β-dihydroequilenin (2 g, 7.45 mmol, clear colorless) in THF (20 ml) at 22° C. (RT) was added Nat-BuO (0.74 g, 7.45 mmol, 97%) in THF (20 ml, hazy solution) dropwise (5 min, temperature raised from 22° C. to 24° C., color changed from clear colorless to dark green solution). After 15 min stirring, at 22° C., sulfur trioxide-triethylamine complex (1.28 g, 7.08 mmol, 0.95 equiv) was added. Reaction was monitored by reverse phase HPLC. After 0.5 h (during 0.5 h dark green reaction mixture changed to light brown) the reaction mixture was concentrated to dryness using a rotary evaporator. To the residual solid was added a solution of tris(hydroxymethyl)aminomethane (TRIS, 1.66 g, assuming 90% conversion by HPLC) in water (200 ml, deionized) and stirred (5 min, hazy light brown colored solution, pH 10-11). The aqueous solution was extracted with diethyl ether (50 ml×3) till no starting diol was seen by HPLC. The aqueous layer (208 g) was partially concentrated (rotary evaporator at 30° C. by 10%, 185 g). Water (20 ml) was added and concentrated (75 g, at around 150 g product slowly crystallizes out). The resulting mixture (light brown) was cooled to 15° C. over 10 min. Stirred at this temperature for 10 min. The slurry was cooled to 8° C. over 10 min. then further cooled to 0-3° C. over another 10 min. Stirred at this temperature for 15 min. Crystals were collected by suction filtration (2.35 g, wet). The solid was dissolved in water (250 ml) containing TRIS (1.45 g, 35% based on the 90% of the product in the HPLC). The solution was clarified by suction filtration, frozen into a shell and lyophilized. 3.78 g, yield 85% (analytical-tris content: 35.59%, KF: 5.55% from Kilo lab).

HPLC (purity area %): 98.4%.

### Example 8

### Preparetion of Sodium-17β-dihydroequilin-3-sulfate

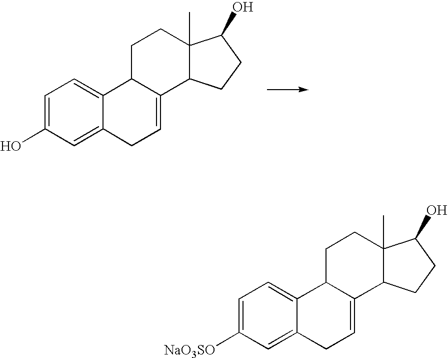

To a solution of 17β-dihydroequilin (3 g, 11.1 mmol) in THF (25 ml) at 22° C. was added a solution of sodium tert-butoxide (1.1 g, 11.1 mmol) in THF (25 ml) dropwise (10 min). After 15 min, sulfur trioxide-triethylamine complex (2.01 g, 11.1 mmol) was added as solid. Reaction was monitored by reverse phase HPLC. After 30 min the solvents were evaporated from the reaction mixture. To the residual solid was added a solution of tris(hydroxymethyl)aminomethane (2.47 g) in water (68 ml, deionized ). The hazy aqueous solution was extracted with diethyl ether (25 ml×4) till no starting material seen in the HPLC. Concentrated the aqueous layer by 10% on a rotary evaporator (to remove any residual ether), added an equivalent amount of water and the concentration was repeated once more. Heated the reaction mixture to 29° C. (to dissolve solids) then cooled down slowly to 3-5° C. After filtration the wet cake (4.35 g) was dissolved in water (60 ml), added tris(hydroxymethyl)aminomethane (2.31 g) and lyophilized to give 5.29 g of the 3-sulfate as a white solid (yield 94%).

¹H-NMR (300 MHz, DMSO-d₆): δ 7.14 (d, 1H, J=8.5 Hz), 6.95 (dd, 1H, J=8.5 & 2.5 Hz), 6.89 (d, 1H, J=2.5 Hz), 5.34 (bs, 1H), 4.62 (d, 1H, J=4.8 Hz), 3.67 (m, 1H), 3.33 (m, 2H), 3.04 (m, 1H), 2.13-2.16 (m. 1H), 1.93-1.97 (m, 2H), 1.81-1.84 (m, 1H), 1.63-1.69 (m, 1H), 1.34-1.45 (m, 4H), 0.53 (s, 3H); TRIS peaks: 4.34 (bh, 3H), 3.21 (bs, 6H), 1.24 (bs, 2H).

¹³C-NMR (300 MHz, DMSO-d₆): δ 151.47, 137.44, 133.53, 132.83, 128.27, 119.94, 119.18, 113.72, 80.53, 49.94, 45.21, 37.55, 32.70, 30.11, 29.57, 20.71, 11.80; TRIS peaks: 63.71, 57.02

ESI MS m/z 349 (M⁻, less Na).

IR (KBr): 3600-3200, 2938, 2876, 1631, 1610, 1589, 1496, 1254, 1050, 1023 cm⁻¹

HPLC (purity area %) 98.2%

### Example 9

### Preparation of Sodium 17β-estradiol-3-sulfate (also know as Sodium 17β-dihydroestrone-3-sulfate)

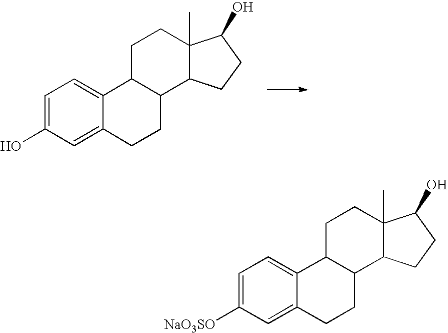

To a solution of 17β-estradiol (also known as 17β-dihydroestrone, 5 g, 18.35 mmol) in THF (40 ml) at 22° C. was added a solution of sodium tert-butoxide (1.76 g, 18.35 mmol) in THF (20 ml) dropwise (2 min). After 15 min, sulfur trioxide-triethylamine complex (2.01 g, 11.1 mmol) was added as solid. Reaction was monitored by reverse phase HPLC. After 30 min the solvents were evaporated from the reaction mixture. To the residual solid was added a solution of tris(hydroxymethyl)aminomethane (4.2 g) in water (200 ml, deionized ). The hazy aqueous solution was extracted with diethyl ether (100 ml and 50 ml×3) till no starting material seen in the HPLC. Concentrated the aqueous layer by 50% on a rotary evaporator (to remove any residual ether), added an equivalent amount of water. Heated the milky reaction mixture to 30° C. (to dissolve), and then lyophilized to give 10.6 g of the 3-sulfate as a white solid (yield 90%).

¹H-NMR (300 MHz, DMSO-d₆): δ 7.15 (1H, d, J=8.4 Hz), 6.87 (2H, m), 4.52 (41H, d, J=4.5 Hz), 3.51 (1H, m), 2.74 (2H, m), 2.28 (1H, m), 2.13 (1H, m), 1.86 (3H, m), 1.6 (1H, m), 1.3 (7H, m): TRIS peaks: 4.34 (bs, 3H), 3.21 (bs, 6H), 1.24 (bs, 2H).

ESI MS m/z 351 (M⁻, less Na).

HPLC (purity area %): 98.6%.

Those skilled in the art will recognize that various changes and/or modifications may be made to aspects or embodiments of this invention and that such changes and/or modifications may be made without departing from the spirit of this invention. Therefore, it is intended that the appended claims cover all such equivalent variations as will fall within the spirit and scope of this invention.

It is intended that each of the patents, applications, and printed publications, including books, mentioned in this patent document be hereby incorporated by reference in their entirety.

What is claimed is:

1. A synthetic process comprising:
reacting a compound of formula IIa: 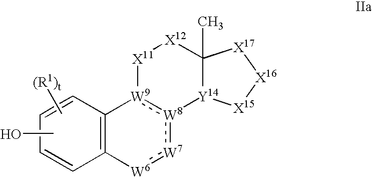
or a salt thereof, wherein:
R¹ is, at each occurrence, independently, halogen, OR^{a}, SR^{a}, —S(═O)R^{a}, —S(═O)₂R^{a}, —NO₂, —NR^{c}R^{d}, —N(R^{c})C(═O)R^{b}, —CN, —CHFCN, —CF₂CN, C₁₋₆ alkyl, C₁₋₆ haloalkyl, C₂₋₇ alkenyl, C₂₋₇ alkynyl, C₃₋₈ cycloalkyl, C₆₋₁₀ aryl, or a 5 or 6-membered heterocyclic ring having 1 to 4 heteroatoms selected from O, N and S, wherein each of the C₁₋₆ alkyl, C₂₋₇ alkenyl and C₂₋₇ alkynyl is optionally substituted by 1, 2, 3, 4 or 5 substituents independently selected from hydroxyl, —CN, —NO₂, halogen, C₁₋₆ haloalkyl, C₁₋₆ alkoxy, C₁₋₆ haloalkoxy, —C(═O)R^{b′}, —C(═O)OR^{a′}, C(═O)NR^{c′}R^{d′}, NR^{c′}R^{d′} and —N(R^{c′})C(═O)R^{b′}; [symbol]
is a single bond or a double bond;
W⁶ and W⁷ are each, independently, CR⁶ or CR⁶R⁷;
R⁶ and R⁷ are each, independently, H, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
W⁸ and W⁹ are each, independently, C or CR⁸;
R⁸ is, at each occurrence, independently, H, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
X¹¹ and X¹² are each, independently, CR¹¹R¹²;
R¹¹ and R¹² are each, independently, H, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
Y⁴ is CR¹⁴;
R¹⁴ is H, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
X¹⁵, X¹⁶ and X¹⁷ are each, independently, CR¹⁵R¹⁶;
R¹⁵ and R¹⁶ are each, independently, hydrogen, hydroxyl, halogen, —CN, —NO₂, C₁₋₆ alkoxy, C₁₋₆ haloalkoxy, C₁₋₆ alkyl, C₁₋₆ haloalkyl, C₂₋₇ alkenyl, C₂₋₇ alkynyl, C₃₋₈ cycloalkyl or C₆₋₁₀ aryl;
R^{a} and R^{b} are each, independently, hydrogen, C₁₋₆ alkyl, C₃₋₈ cycloalkyl or C₆₋₁₀ aryl;
R^{a′} and R^{b′} are each, independently, hydrogen, C₁₋₆ alkyl, C₃₋₈ cycloalkyl or C₆₋₁₀ aryl;
R^{c} and R^{d} are each, independently, hydrogen, C₁₋₆ alkyl, C₃₋₈ cycloalkyl or C₆₋₁₀ aryl;
or R^{c} and R^{d} together with the N atom to which they are attached form a 4-, 5-, 6- or 7-membered heterocycloalkyl group;
R^{c′} and R^{d′} are each, independently, hydrogen, C₁₋₆ alkyl, C₃₋₈ cycloalkyl or C₆₋₁₀ aryl;
or R^{c′} and R^{d′} together with the N atom to which they are attached form a 4-, 5-, 6- or 7-membered heterocycloalkyl group; and
t is 0, 1, 2, or 3,
provided that at least one of X¹⁵, X¹⁶ and X¹⁷ is C(OH)R¹⁶,
with a sulfating reagent in the presence of a base having the structure of ML wherein M is an alkali metal ion; and L is hydride (H⁻), hydroxide (OH⁻), or C₁₋₁₀ alkoxide (C₁₋₁₀ alkyl-O⁻),
for a time and under conditions sufficient to form a compound of Formula Ia: 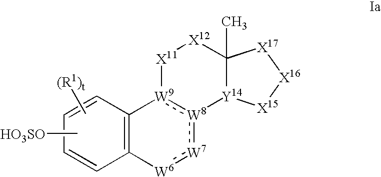
or an alkali metal salt thereof, wherein the product of the reaction of the process is substantially free of a compound of Formula XX: 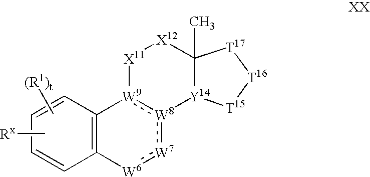
or a salt thereof, wherein R^{x} is OH or OSO₃H; and T¹⁵, T¹⁶ and T¹⁷ are each, independently, CR¹⁵R¹⁶ or C(OSO₃H)R¹⁶, and at least one of T¹⁵, T¹⁶ and T¹⁷ is C(OSO₃H)R¹⁶.

2. The synthetic process of claim 1 wherein:
Formula IIa is Formula IIaa: 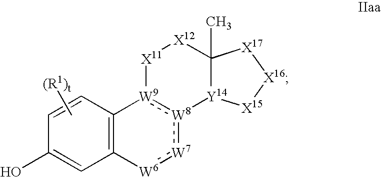
and Formula Ia is Formula Iaa:

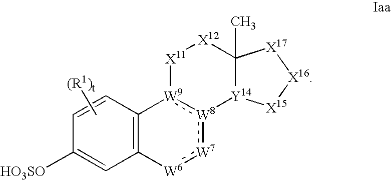

3. The synthetic process of claim 1 wherein:
Formula IIa is Formula IIab: 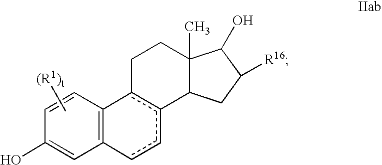
and Formula Ia is Formula Iab:

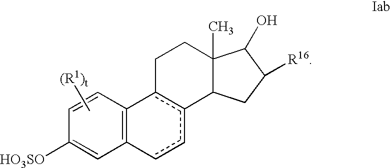

4. The synthetic process of claim 1 wherein M is Li⁺, Na⁺ or K⁺.

5. The synthetic process of claim 1 wherein L is hydride or C₁₋₁₀ alkoxide.

6. The synthetic process of claim 1 wherein ML is Na⁺ (C₁₋₁₀ alkoxide) or K⁺ (C₁₋₁₀ alkoxide).

7. The synthetic process of claim 6 wherein ML is Na⁺ (C₁₋₄ alkoxide) or K⁺ (C₁₋₄ alkoxide).

8. The synthetic process of claim 1 wherein the amount of the base is about 0.95 to about 1.05 molar equivalents to the compound of Formula IIa, or the salt thereof.

9. The synthetic process of claim 8 wherein the amount of the base is about one molar equivalent to the compound of Formula IIa, or the salt thereof.

10. The synthetic process of claim 1 wherein the sulfating reagent comprises a complex of sulfur trioxide and a tertiary amine; or a complex of sulfur trioxide and an amide.

11. The synthetic process of claim 10 wherein the sulfating reagent comprises a complex of sulfur trioxide and a tertiary amine; and wherein the tertiary amine is selected from trialkylamine and pyridine.

12. The synthetic process of claim 11 wherein the sulfating reagent comprises a complex of sulfur trioxide and triethylamine.

13. The synthetic process of claim 1 wherein the base is mixed with the compound of Formula IIa or the salt thereof before the reacting the compound of Formula IIa or the salt thereof with the sulfating reagent.

14. The synthetic process of claim 1 wherein the reacting the compound of Formula IIa or the salt thereof with the sulfating reagent is performed in a solvent system.

15. The process of claim 14 wherein the solvent system comprises a polar aprotic organic solvent.

16. The process of claim 15 wherein the solvent system comprises one or more of an ether, an ester, an alcohol, an alkylnitrile, and a halogenated hydrocarbon.

17. The process of claim 15 wherein the solvent system comprises one or more of tetrahydrofuran, 2-methyl-tetrahydrofuran, acetonitrile, N N-dimethylformamide, N,N-dimethylacetamide, N-methyl-2-pyrrolidone, methylene chloride, and chloroform.

18. The process of claim 1 wherein the reacting the compound of Formula IIa or the salt thereof with the sulfating reagent is performed at a temperature of less than about 100° C.

19. The process of claim 18 wherein the reacting the compound of Formula IIa or the salt thereof with the sulfating reagent is performed at a temperature of from about 20° C. to about 60° C.

20. The process of claim 1 further comprising isolating the compound of Formula Ia or the salt thereof and optionally purifying the isolated compound of Formula Ia or the salt thereof.

21. The process of claim 1 further comprising adding tris(hydroxymethyl)aminomethane to the compound of Formula Ia or the salt thereof.

22. The process of claim 21 further comprising isolating a composition which comprises tris(hydroxymethyl)aminomethane and the compound of Formula Ia or the salt thereof.

23. The process of claim 22 wherein the process is carried out in one reaction vessel.

24. A compound which is an alkali metal salt of estra(1,3,5-triene)-3,16β,17α-triol-3-sulfate.

25. A compound which is sodium estra(1,3,5-triene)-3,16β,17α-triol-3-sulfate.

26. A composition comprising an alkali metal salt of estra(1,3,5-triene)-3,16β,17α-triol-3-sulfate.

27. A composition comprising an alkali metal salt of estra(1,3,5-triene)-3,16β,17α-triol-3-sulfate and tris(hydroxymethyl)aminomethane, wherein the composition is substantially free from other estrogenic steroids; or a composition comprising an alkali metal salt of 17β-dihydroequilenin-3-sulfate and tris(hydroxymethyl)aminomethane, wherein the composition is substantially free from other estrogenic steroids; or a composition comprising an alkali metal salt of 17β-dihydroequilin-3-sulfate and tris(hydroxymethyl)aminomethane, wherein the composition is substantially free from other estrogenic steroids; or a composition comprising an alkali metal salt of 17β-estradiol-3-sulfate and tris(hydroxymethyl)aminomethane, wherein the composition is substantially free from other estrogenic steroids.

28. The synthetic process of claim 1 wherein the compound of Formula IIa is 17α-estradiol; the alkali metal salt of the compound of Formula Ia is sodium 17α-estradiol-3-sulfate; and the sulfating reagent comprises a complex of sulfur trioxide and triethylamine; or
the synthetic process of claim 1 wherein the compound of Formula IIa is 17β-estradiol; the alkali metal salt of the compound of Formula Ia is sodium 17β-estradiol-3-sulfate; and the sulfating reagent comprises a complex of sulfur trioxide and triethylamine; or
the synthetic process of claim 1 wherein the compound of Formula IIa is 17β-dihydroequilenin; the alkali metal salt of the compound of Formula Ia is sodium 17β-dihydroequilenin-3-sulfate; and the sulfating reagent comprises a complex of sulfur trioxide and triethylamine; or
the synthetic process of claim 1 wherein the compound of Formula IIa is 17β-dihydroequilin; the alkali metal salt of the compound of Formula Ia is sodium 17β-dihydroequilin-3-sulfate; and the sulfating reagent comprises a complex of sulfur trioxide and triethylamine; or
the synthetic process of claim 1 wherein the compound of Formula IIa is 17α-dihydroequilin; the alkali metal salt of the compound of Formula Ia is sodium 17α-dihydroequilin-3-sulfate; and the sulfating reagent comprises a complex of sulfur trioxide and triethylamine; or
the synthetic process of claim 1 wherein the compound of Formula IIa is estra(1,3,5-triene)-3,16β,17α-triol; the alkali metal salt of the compound of Formula Ia is sodium-estra(1,3,5-triene)-3,16β,17α-triol-3-sulfate; and the sulfating reagent comprises a complex of sulfur trioxide and triethylamine.

29. The process according to claim 28 further comprising adding tris(hydroxymethyl)aminomethane to the alkali metal salt of the compound of Formula Ia and optionally isolating a composition which comprises tris(hydroxymethyl)aminomethane and the alkali metal salt of the compound of Formula Ia.

30. The product of the process according to claim 29.

31. A method of treating a mammal having a disease or syndrome associated with estrogen deficiency or excess of estrogen comprising administering to said mammal a therapeutically effective amount of the compound of claim 24.

32. A method of treating a mammal having a disease or disorder associated with proliferation or abnormal development of endometrial tissues comprising administering to said mammal a therapeutically effective amount of the compound of claim 24.

33. A method of lowering cholesterol in a mammal comprising administering to said mammal a therapeutically effective amount of the compound of claim 24.

34. A method of treating a postmenopausal woman for one or more vasomotor disturbances comprising administering to said postmenopausal woman a therapeutically effective amount of the compound of claim 24.

35. The method of claim 34 wherein the vasomotor disturbance is hot flush.

36. A method of inhibiting bone loss in a mammal, comprising administering to said mammal a therapeutically effective amount of the compound of claim 24.

37. A method of treating breast cancer in a mammal, comprising administering to said mammal a therapeutically effective amount of the compound of claim 24.

38. A method of treating a mammal having a disease or syndrome associated with estrogen deficiency or excess of estrogen comprising administering to said mammal a therapeutically effective amount of the composition according to claim 27.

39. A method of treating a mammal having a disease or disorder associated with proliferation or abnormal development of endometrial tissues comprising administering to said mammal a therapeutically effective amount of the composition according to claim 27.

40. A method of lowering cholesterol in a mammal comprising administering to said mammal a therapeutically effective amount of the composition according to claim 27.

41. A method of treating a postmenopausal woman for one or more vasomotor disturbances comprising administering to said postmenopausal woman a therapeutically effective amount of the composition according to claim 27.

42. The method of claim 41 wherein the vasomotor disturbance is hot flush.

43. A method of inhibiting bone loss in a mammal, comprising administering to said mammal a therapeutically effective amount of the composition according to claim 27.

44. A method of treating breast cancer in a mammal, comprising administering to said mammal a therapeutically effective amount of the composition according to claim 27.
